(19) United States
(12) Patent Application Publication
(10) Pub. No.: US 2004/0044082 A1
(43) Pub. Date: Mar. 4, 2004

(54) Topical composition and method for treating urinary stress incontinence

(76) Inventor: Michael G. Wyllie, Kent (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/450,435

(22) PCT Filed: Dec. 17, 2001
(86) PCT No.: PCT/GB01/05545

(30) Foreign Application Priority Data
Dec. 15, 2000 (GB) 0030580.5

(51) Int. Cl. A61K 31/137

(52) U.S. Cl. 514/649

(57) ABSTRACT

There is provided a method and composition for treating urinary stress incontinence based upon the topical administration of an alpha-adrenoceptor agonist, such as phenylephrine. A daily dosage of 20 mg to 2000 mg is suitable, and the composition may conveniently be applied as a cream or gel to the vaginal or peri-urethral area.

The present invention relates to a pharmaceutical composition for treating urinary stress incontinence.

Urinary incontinence is the involuntary loss of urine which is objectively demonstrable, and the condition presents both a social or hygienic problem to those affected. “Stress incontinence” is the term used to describe the condition when the involuntary loss of urine occurs during physical exertion. Stress incontinence will affect up to 10% of the adult female population. The incidence increases substantially after child-birth.

Conventionally, treatment of urinary stress incontinence has included either lifestyle modification or surgery. The former treatment includes weight reduction, reduction or elimination of smoking and reduction of food, and particularly fluid, intake. Various surgical procedures are also possible, should the symptoms be sufficiently severe. The use of drugs is not widespread, and sufferers often have to resort to the use of incontinence pads and adult nappies.

Many factors appear to be involved in the pathogenesis of urinary stress incontinence, including urethral support, bladder neck function and the tone and function of the urethral muscle. Women with stress incontinence have lower resting urethral pressures than age matched controls not experiencing symptoms. It is logical therefore that an increased urethral pressure should alleviate the condition.

There is ample pharmacological evidence that a substantial part of urethral tone and thereby urethral pressure is mediated through stimulation of alpha 1-adrenoceptors (Andersson (1993) Pharmaco Rev 45; 253-308). Drug therapy has been directed towards increasing tone in the urethral muscle by mimicking the action of the natural neurotransmitter, noradrenaline, on the alpha 1-adrenoceptors. There is clinical data on several of these noradrenaline mimics or alpha-adrenoceptor agonists including ephedrine and norephedrine (Andersson (1988) Drugs 35; 477-494; Wein (1995) Urol Clin N Am 22; 557-577) and indeed phenylephrine (Schreiter et al (1976) Urol Int 31; 13-18). However, although these agents can produce modest benefit, their clinical use is limited by obtrusive increases in blood pressure (Thomas et al (1991) Br J Clin Pharmacol 32: 705-711) and cardiovascular side effects such as dizziness, headaches and tremors (Andersson (1999) Int Consultation in Incontinence, Plymbridge Distributors ed Abrams, Khoury and Wein: 449-486). Although attempts have been made to synthesise improved alpha-adrenoceptor agonists, this class of agent has not been established in the management of urinary stress incontinence. It has also not been possible to find drug doses that can discriminate between the unwanted cardiovascular effects and the beneficial effect on the urethra.

Phenylephrine is an example of an alpha-adrenoceptor agonist. However, it is unsuitable for the oral treatment of stress incontinence. Phenylephrine has also been shown to produce substantial elevations in blood pressure and it has low oral bioavailability and a short duration of action which would render it useless as a potential oral therapeutant (Thomas et al (1991) Br J Clin Pharmacol 32: 705-7). Phenylephrine, however, is used clinically as a topical agent for the treatment of certain ophthalmological conditions or as a topical anaesthetic agent (Hoffman and Lefkowitz (1996) in Goodman and Gilman's The Pharmacological Basis of Therapeutics (9^{th} edition), McGraw-Hill ed by Hardman et al: 199-248).

To date, there has been no disclosure of the use of topical alpha-adrenoceptor agonists, such as phenylephrine, alone or in combination, for the treatment of urinary stress incontinence.

It is an object of the present invention to consider the topical application of alpha-adrenoceptor agonists, especially alpha 1-adrenoceptor agonists such as phenylephrine, to patients experiencing symptoms of urinary stress incontinence. Delivery in this way will result in a considerable improvement of the benefit-risk profile of the active agents. It is anticipated that this route will obtain regulatory approval as a prescription-only medication.

The present invention thus provides a composition for treating urinary stress incontinence, said composition comprising an alpha-adrenoceptor agonist (especially phenylephrine) as an active ingredient, together with a pharmaceutically acceptable carrier or excipient. The composition is desirably formulated to be suitable for topical application, for example to vaginal tissue.

In a further aspect, the present invention provides the use of an alpha-adrenoceptor agonist (for example phenylephrine) in the manufacture of a medicament for treating urinary stress incontinence via topical application, for example to vaginal or peri-urethral tissue.

In a yet further aspect, the present invention provides a method of treating urinary stress incontinence in female patients, said method comprising the topical application of an alpha-adrenoceptor agonist (preferably phenylephrine) to vaginal or peri-urethral tissue of said patient.

Thus, according to the present invention urinary stress incontinence will be treated by local administration to vaginal tissue compositions containing the alpha-adrenoceptor agonist, phenylephrine. In this way the major obstacles to the routine clinical use of phenylephrine in the treatment of stress incontinence, such as blood pressure elevation and short duration of action, will be circumvented.

As used herein “stress incontinence” includes all types of urinary incontinence arising exclusively, or in part, from physical exertion or provocation. Clinically, this will include mild, moderate and severe symptoms arising from genuine stress incontinence or secondary to mixed (stress-urge) incontinence, irrespective of the method of diagnosis. In particular the invention will be of benefit to women unsuitable for, or unwilling to undergo, surgery and/or unwilling to use incontinence pads or adult nappies.

It is preferred that the urinary stress incontinence is treated by local or topical application of the pharmaceutical composition in, onto or around the area of the vagina and/or vaginal mucosa, in close proximity to the urethra. A method for such topical delivery is described by Hilton and Stanton 1993 (Br J Obstet Gynecol 90; 940-944). In all cases a pharmaceutically acceptable carrier or excipient will be present with the active moiety.

The pharmaceutical compositions for topical delivery may be formulated as creams, ointments, suspensions, lotions, powders, gels, solutions, pastes, controlled or slow releasing matrices or depots, sprays, foams, oils, suppositories, enemas, or drug delivery devices.

The topical compositions can comprise emulsifiers, preservatives, stabilising and pH buffering agents and anti-oxidants. The compositions may also preferably comprise steroids with oestrogenic activity.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, Mack Publishing Company, Easter Pa., 15^{th} edition (1975).

The exact dosage of phenylephrine in the administered composition will depend on the subject being treated, on the severity of the stress incontinence being treated, the age weight and medical condition of the patient being treated and ultimately on the judgement of the prescribing physician. Thus because of the patient-to-patient variation and the variability of the condition form day-to-day, the dosages given below are guidelines only, and the physician may adjust the dose of phenylephrine to achieve the response considered appropriate for the patient.

In general for a 70 kg patient with moderate stress urinary incontinence the dose of alpha-adrenoceptor agonist (such as phenylephrine) would be in the range 20 mg to 2000 mg per day, preferably at 50 mg to 200 mg per day.

The percentage active ingredient (preferably phenylephrine) in the composition is preferably at least 5% w/w, more preferably at least 10% w/w, and advantageously up to 50% w/w of the whole composition. Exemplary compositions may comprise 15 to 25% w/w pherylephrine, for example 20% w/w phenylephrine. The dosage of phenylephrine will be at least 20 mg per 0.5 ml of the composition, more preferably at least 50 mg per 0.5 ml and advantageously up to 200 mg per 0.5 ml. The topical application of the composition should be between 1 and 6 times daily, for example 3 times a day, or at frequencies providing adequate relief of the symptoms of urinary stress incontinence.

The composition, in addition to phenylephrine, may comprise tissue membrane penetration enhancing agents, particularly sulphoxides, such as dimethylsulphoxide (DMSO), preferably within the range 25 to 50% w/w. Suitable alternatives are amides (DMA, DMF), pyrrolidines, organic solvents and calcium thioglycollate and polyacrylic acid derivatives. Several of the constituents would act to aid penetration by actions as tissue hydrating agents and/or emusifiers. Other agents, particularly, propylene glycol, may also be present to soften the skin. The final composition will have a slightly acidic pH, preferably in the range 3.5 to 4.5.

The present invention will be further described by reference to the non-limiting example and figures, in which:

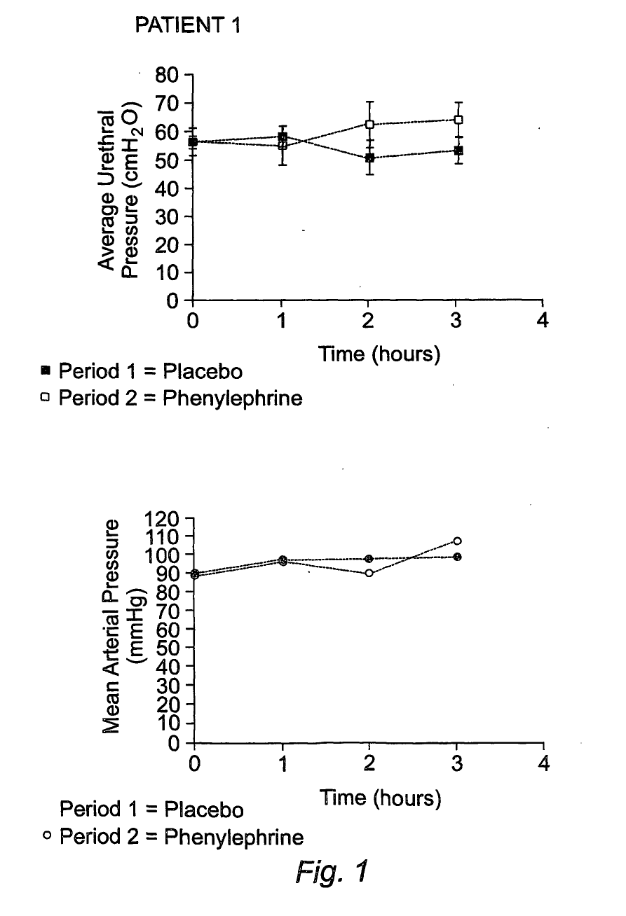

FIG. 1 represents the average urethral pressure over time and the mean arteial pressure over time for a first patient. ▪=period 1, placebo; □=period 2, phenylephrine; ●=period 1, placebo; ◯=period 2, phenylephrine.

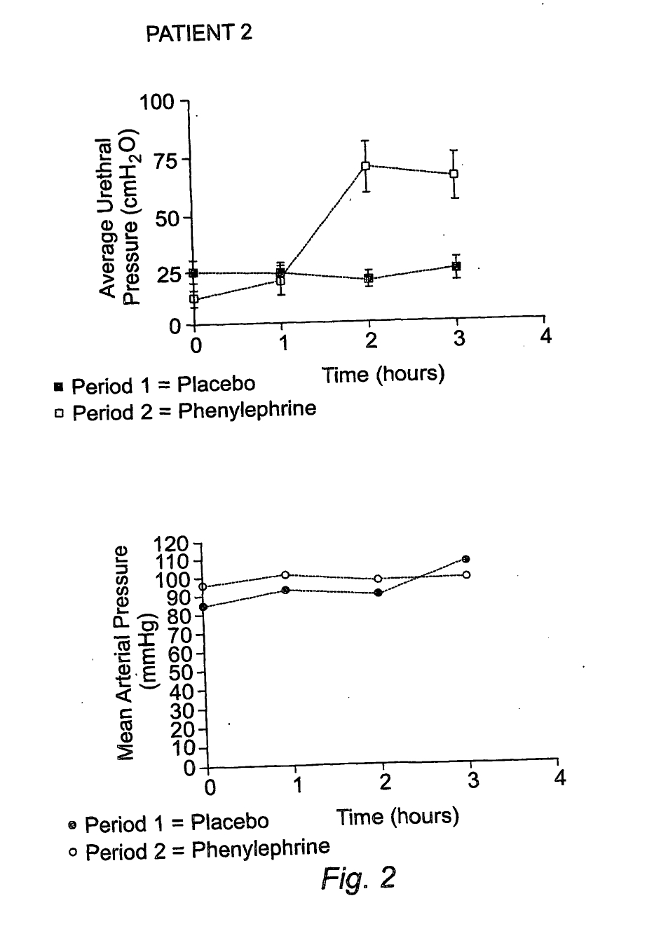

FIG. 2 represents the average urethral pressure over time and the mean arteial pressure over time for a second patient. ▪=period 1, placebo; □=period 2, phenylephrine; ●=period 1, placebo; ◯=period 2, phenylephrine.

### EXAMPLE

The invention has been evaluated in a double-blind, randomised, 2-way cross-over study in female patients with documented history of stress incontinence. Each patient was exposed to both placebo and active drug over the course of the study.

Each patient received either a single dose of topical phenylephrine gel (20% w/w in a volume of 0.5 ml) or placebo according to the randomisation schedule. The gel was applied to the peri-urethral area; in, onto or around the area of the vagina and/or vaginal mucosa, in close proximity to the urethra. For up to 5 hours post dosing, urethral pressure was measured using the standard Lectromed/Galetec MPR/2 ambulatory measurement system. Supine and standing blood pressure and pulse rate was likewise measured using standard methodology via a Dinamap semi-automatic sphygmomanometer.

The results obtained from two typical patients (FIGS. 1 and 2) show that topical phenylephrine (0.5 ml, 20% w/w), in contrast to placebo, produces marked and sustained increases in intra-urethral pressure. In contrast to the well-documented changes in blood pressure observed after systemic administration, topical phenylephrine produced no clinically significant changes in diastolic or systolic blood pressure or in pulse rate.

1. A composition for treating urinary stress incontinence, said composition comprising an alpha-adrenoceptor agonist as an active ingredient, together with a pharmaceutically acceptable carrier or excipient.

2. A composition as claimed in claim 1 for topical application.

3. A composition as claimed in either one of claims 1 and 2 wherein said alpha-adrenoceptor agonist is an alpha-1-adrenoceptor agonist.

4. A composition as claimed in claim 3 wherein said alpha-1-adrenoceptor agonist is phenylephrine.

5. A composition as claimed in any one of claims 1 to 4 in the form of a cream, ointment, suspension, lotion, powder, gel, solution, paste, spray, foam, oil, suppository, enema, controlled or slow release matrix or depot or a drug delivery device.

6. A composition as claimed in any one of claims 1 to 5 which comprises at least 5% w/w active ingredient.

7. The use of an alpha-adrenoceptor agonist in the manufacture of a medicament for treating urinary stress incontinence via topical application.

8. The use as claimed in claim 7 wherein said alpha adrenoceptor agonist is an alpha-1-adrenoceptor agonist.

9. The use as claimed in claim 8 wherein said alpha 1-adrenoceptor agonist is phenylephrine.

10. The use as claimed in any one of claims 7 to 9 wherein said medicament is in the form of a cream, ointment, suspension, lotion, powder, gel, solution, paste, spray, foam, oil, suppository, enema, controlled or slow release matrix or depot or a drug delivery device.

11. The use as claimed in any one of claims 7 to 10 wherein said medicament comprises at least 5% w/w active ingredient.

12. A method of treating urinary stress incontinence in a female patient, said method comprising the topical application of an alpha-adrenoceptor agonist to the vaginal or peri-urethral tissue of said patient.

13. The method as claimed in claim 12 wherein said alpha-adrenoceptor agonist is an alpha-1-adrenoceptor agonist.

14. The method is claimed in claim 13, wherein said alpha-1-adrenoceptor agonist is phenylephrine.

15. The method as claimed in any one of claims 12 to 14 wherein said alpha-adrenoceptor agonist is in the form of a cream, ointment, suspension, lotion, powder, gel, solution, paste, spray, foam, oil, suppository, enema, controlled or slow release matrix or depot or a drug delivery device.

16. The method as claimed in any one of claims 12 to 15 wherein the dose of said alpha-adrenoceptor agonist is 20 mg to 2000 mg per day.
